(19) United States
(12) Patent Application Publication
(10) Pub. No.: US 2018/0296320 A1
(43) Pub. Date: Oct. 18, 2018

(54) FORECASTING CATARACT SURGERY EFFECTIVENESS

(71) Applicant: Microsoft Technology Licensing, LLC, Redmond, WA (US)

(72) Inventors: Manish Gupta, Hyderabad (IN); Vinay Vemula, Hyderabad (IN); Prashant Gupta, Hyderabad (IN); Raghuram Lanka, Hyderabad (IN)

(21) Appl. No.: 15/630,962

(22) Filed: Jun. 23, 2017

(30) Foreign Application Priority Data
Apr. 14, 2017 (IN) 201741013415

(51) Int. Cl. A61F 2/16; A61B 3/10

(52) U.S. Cl. CPC A61F 2/16; A61B 3/0025; A61F 2/1605; A61B 3/10

(57) ABSTRACT

Aspects extend to methods, systems, and computer program products for forecasting cataract surgery effectiveness. Medical practitioners can use a predictive model to automatically forecast cataract surgery effectiveness for patients, including predicting both refractive outcomes (cylinder and sphere) and visual acuity outcomes (UCVA and BCVA), recommending a cataract surgery type, and recommending an IOL type and power. The predictive model can be offered to medical practitioners as a Web API, as an application on the web, as a SaaS offering, as an application on mobiles or medical devices, or any number of other platforms. Patients can be ranked based on predicted cataract surgery outcomes. The rankings can be used to better allocate limited medical resources to patients deriving more benefit.

### CROSS-REFERENCE TO RELATED APPLICATIONS

This application claims priority to Indian National Application No. 201741013415, filed Apr. 14, 2017, and entitled “FORECASTING CATARACT SURGERY EFFECTIVENESS”.

### BACKGROUND

### 1. Background and Relevant Art

Cataract is the opacification of the lens of the eye, which initially prevents clear vision and eventually progresses to blindness if left untreated. It is estimated that nearly half of the millions of cases of bilateral blindness worldwide are caused by cataract. Cataract surgery is the replacement of the natural (crystalline) lens of the eye that has developed a cataract with an artificial lens.

There are a wide variety of different types of cataract surgery that can be performed. Often, any of several different types of cataract surgery can be indicated for cataract. Each eye surgeon can prefer particular types of cataract surgery over others and selection of cataract surgery type lacks consistency.

### BRIEF SUMMARY

Examples extend to methods, systems, and computer program products for forecasting cataract surgery effectiveness. A computer system accesses eye characteristic data for the patient's eyes. The eye characteristic data is taken from diagnostic procedures performed on the patient. The eye characteristic data indicates that vision in at least one of the patient's eyes is at least partially impaired due to cataract. The computer system accesses demographic data for the patient.

The computer system inputs the eye characteristic data and demographic data into a predictive model in system memory. The predictive model is formulated from surgery data for a plurality of previously performed cataract surgeries. The surgery data includes, for each of the plurality of previous performed cataract surgeries, one or more of: patient pre-operative eye characteristic data, patient demographic data, a cataract surgery type, interocular lens features, one or more patient post-operative outcomes, and a time difference between the time of cataract surgery and a post-operative exam when the one or more post-operative outcomes were detected. For each of the plurality of previously performed cataract surgeries, the predictive model transforms the one or more of: the patient pre-operative eye characteristic data, the patient demographic data, the cataract surgery type, the interocular lens features, the one or more patient post-operative outcomes, and the time difference through regression analysis.

The predictive model uses regression analysis to forecast the effectiveness of one or more different types of cataract surgery in combination with one or more different interocular lens types and powers for the patient. The effectiveness for each of one or more different types of cataract surgeries in combination with one or more different interocular lens types and powers is indicated by predicted positive outcomes for one or more of: patient refraction and patient visual acuity for the patient. Predicted positive outcomes are inferred based on the patient's eye characteristic data and demographic data in view of surgery data from previous cataract surgeries.

The predictive model matches the patient to a selected cataract surgery type, interocular lens type, and interocular lens power based on the predicted positive outcomes for the patient. The computer system returns the selected cataract surgery type, interocular lens type, interocular lens power, and predicted positive outcomes as a forecast.

Predicted positive outcomes for a plurality of patients can be forecasted. Patients can be ranked relative to one another based on predicted positive outcomes. Patients with a higher likelihood of improving sight or having a likelihood of more significant sight improvement via cataract surgery can be ranked higher than other patients with a lower likelihood of improving sight or having a likelihood of less significant sight improvement. When limited resources are available to perform cataract surgeries, the limited resources can be allocated to higher ranked patients.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by practice. The features and advantages may be realized and obtained by means of the instruments and combinations particularly pointed out in the appended claims. These and other features and advantages will become more fully apparent from the following description and appended claims, or may be learned by practice as set forth hereinafter.

### BRIEF DESCRIPTION OF THE DRAWINGS

In order to describe the manner in which the above-recited and other advantages and features can be obtained, a more particular description will be rendered by reference to specific implementations thereof which are illustrated in the appended drawings. Understanding that these drawings depict only some implementations and are not therefore to be considered to be limiting of its scope, implementations will be described and explained with additional specificity and detail through the use of the accompanying drawings in which:

FIG. 1 illustrates an example computer architecture that facilitates formulating and training a predictive model to forecast cataract surgery effectiveness.

FIG. 2 illustrates an example computer architecture that facilitates forecasting cataract surgery effectiveness.

FIG. 3 illustrates a flow chart of an example method for forecasting cataract surgery effectiveness.

### DETAILED DESCRIPTION

Examples extend to methods, systems, and computer program products for forecasting cataract surgery effectiveness. A computer system accesses eye characteristic data for the patient's eyes. The eye characteristic data is taken from diagnostic procedures performed on the patient. The eye characteristic data indicates that vision in at least one of the patient's eyes is at least partially impaired due to cataract. The computer system accesses demographic data for the patient.

The computer system inputs the eye characteristic data and demographic data into a predictive model in system memory. The predictive model is formulated from surgery data for a plurality of previously performed cataract surgeries. The surgery data includes, for each of the plurality of previous performed cataract surgeries, one or more of: patient pre-operative eye characteristic data, patient demographic data, a cataract surgery type, interocular lens features, one or more patient post-operative outcomes, and a time difference between the time of cataract surgery and a post-operative exam when the one or more post-operative outcomes were detected. For each of the plurality of previously performed cataract surgeries, the predictive model transforms the one or more of: the patient pre-operative eye characteristic data, the patient demographic data, the cataract surgery type, the interocular lens features, the one or more patient post-operative outcomes, and the time difference through regression analysis.

The predictive model uses regression analysis to forecast the effectiveness of one or more different types of cataract surgery in combination with one or more different interocular lens types and powers for the patient. The effectiveness for each of one or more different types of cataract surgeries in combination with one or more different interocular lens types and powers is indicated by predicted positive outcomes for one or more of: patient refraction and patient visual acuity for the patient. Predicted positive outcomes are inferred based on the patient's eye characteristic data and demographic data in view of surgery data from previous cataract surgeries.

The predictive model matches the patient to a selected cataract surgery type, interocular lens type, and interocular lens power based on the predicted positive outcomes for the patient. The computer system returns the selected cataract surgery type, interocular lens type, interocular lens power, and predicted positive outcomes as a forecast.

Predicted positive outcomes for a plurality of patients can be forecasted. Patients can be ranked relative to one another based on predicted positive outcomes. Patients with a higher likelihood of improving sight or having a likelihood of more significant sight improvement via cataract surgery can be ranked higher than other patients with a lower likelihood of improving sight or having a likelihood of less significant sight improvement. When limited resources are available to perform cataract surgeries, the limited resources can be allocated to higher ranked patients.

Implementations may comprise or utilize a special purpose or general-purpose computer including computer hardware, such as, for example, one or more computer and/or hardware processors (including Central Processing Units (CPUs) and/or Graphical Processing Units (GPUs)) and system memory, as discussed in greater detail below. Some computer systems can include and/or be (e.g., network) connected to eye examination devices for examining and/or mapping the human eye. Other devices are also discussed in greater detail below.

Implementations also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, implementations can comprise at least two distinctly different kinds of computer-readable media: computer storage media (devices) and transmission media.

Computer storage media (devices) includes RAM, ROM, EEPROM, CD-ROM, Solid State Drives (“SSDs”) (e.g., RAM-based or Flash-based), Shingled Magnetic Recording (“SMR”) devices, Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

In one aspect, one or more processors are configured to execute instructions (e.g., computer-readable instructions, computer-executable instructions, etc.) to perform any of a plurality of described operations. The one or more processors can access information from system memory and/or store information in system memory. The one or more processors can (e.g., automatically) transform information between different formats, such as, for example, between any of: surgery data, eye characteristics, demographics, keratometry, surgery types, interocular lens (IOL) features, doctor identifiers, surgically induced astigmatism (SIA) values, time differences, other eye conditions, family history, prior surgeries, cataract types, interocular lens (IOL) placement, predicted and measured post-operative sphere, predicted and measured post-operative cylinder, predicted and measured post-operative Best Corrected Visual Acuity (BCVA), predicted and measured patient post-operative Uncorrected Visual Acuity (UCVA), transformed surgery data, filled-in surgery data, training data, predictive models, outcome maps, recommendations, forecasted cataract surgery effectiveness, patient rankings, etc.

System memory can be coupled to the one or more processors and can store instructions (e.g., computer-readable instructions, computer-executable instructions, etc.) executed by the one or more processors. The system memory can also be configured to store any of a plurality of other types of data generated and/or transformed by the described components, such as, for example, surgery data, eye characteristics, demographics, keratometry, surgery types, interocular lens (IOL) features, doctor identifiers, surgically induced astigmatism (SIA) values, time differences, other eye conditions, family history, prior surgeries, cataract types, interocular lens (IOL) placement, predicted and measured post-operative sphere, predicted and measured post-operative cylinder, predicted and measured post-operative Best Corrected Visual Acuity (BCVA), predicted and measured patient post-operative Uncorrected Visual Acuity (UCVA), transformed surgery data, filled-in surgery data, training data, predictive models, outcome maps, recommendations, forecasted cataract surgery effectiveness, patient rankings, etc.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to computer storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that computer storage media (devices) can be included in computer system components that also (or even primarily) utilize transmission media.

Computer-executable instructions comprise, for example, instructions and data which, in response to execution at a processor, cause a general purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the described aspects may be practiced in network computing environments with many types of computer system configurations, including, personal computers, desktop computers, laptop computers, message processors, hand-held devices, wearable devices, multicore processor systems, multi-processor systems, microprocessor-based or programmable consumer electronics, network PCs, minicomputers, mainframe computers, mobile telephones, PDAs, tablets, routers, switches, and the like. The described aspects may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Further, where appropriate, functions described herein can be performed in one or more of: hardware, software, firmware, digital components, or analog components. For example, one or more application specific integrated circuits (ASICs) can be programmed to carry out one or more of the systems and procedures described herein. In another example, computer code is configured for execution in one or more processors, and may include hardware logic/electrical circuitry controlled by the computer code. These example devices are provided herein purposes of illustration, and are not intended to be limiting. Embodiments of the present disclosure may be implemented in further types of devices.

The described aspects can also be implemented in cloud computing environments. In this description and the following claims, “cloud computing” is defined as a model for enabling on-demand network access to a shared pool of configurable computing resources. For example, cloud computing can be employed in the marketplace to offer ubiquitous and convenient on-demand access to the shared pool of configurable computing resources (e.g., compute resources, networking resources, and storage resources). The shared pool of configurable computing resources can be provisioned via virtualization and released with low effort or service provider interaction, and then scaled accordingly.

A cloud computing model can be composed of various characteristics such as, for example, on-demand self-service, broad network access, resource pooling, rapid elasticity, measured service, and so forth. A cloud computing model can also expose various service models, such as, for example, Software as a Service (“SaaS”), Platform as a Service (“PaaS”), and Infrastructure as a Service (“IaaS”). A cloud computing model can also be deployed using different deployment models such as private cloud, community cloud, public cloud, hybrid cloud, and so forth. In this description and in the following claims, a “cloud computing environment” is an environment in which cloud computing is employed. Aspects of the invention can be implemented in a cloud environment.

In this description and the following claims, “cataract” is defined as a clouding of the lens in the eye which leads to a decrease in vision. Clumps of protein or yellow-brown pigment may be deposited in the lens reducing the transmission of light to the retina at the back of the eye. Cataracts can affect one or both eyes. Symptoms may include faded colors, blurry vision, halos around light, trouble with bright lights, trouble seeing at night, and blindness. Cataracts can be caused by aging, due to trauma or radiation exposure, be present from birth, or occur following eye surgery for other conditions.

In this description and the following claim, “cataract surgery” is defined as any surgical procedure for correcting loss of vision due to cataract. Cataract surgery is defined to include: extracapsular cataract extraction (ECCE), intrascapular cataract extraction (ICEE), Small incision cataract surgery (SICS), phacoemulsification (or “PHACO”) cataract surgery with foldable intraocular lens (IOL) implantation and laser assisted cataract surgery.

Phaco cataract surgery includes making a relatively small incision (a “side port”) in the eye. A high-frequency ultrasound device is inserted into the eye through the incision. The high-frequency ultrasound device breaks up a cloudy lens into small pieces, which are then removed from the eye with suction. A foldable clear interocular lens is inserted through the incision. Once in the eye, the clear interocular lens is unfolded and positioned behind the iris and pupil in essentially the same location the natural lens occupied. Due the small size of the incision, stitches are usually not used.

Extracapsular cataract extraction (ECCE) includes making a larger incision in the eye relative to Phaco cataract surgery. ECCE involves removal of almost the entire natural lens while the elastic lens capsule (posterior capsule) is left intact to allow implantation of an intraocular lens. It involves manual expression of the lens through a large (e.g., 1012 mm) incision made in the cornea or sclera. A clear interocular lens is then inserted through the incision and positioned behind the iris and pupil in essentially the same location the natural lens occupied. The incision can then be stitched closed. ECCE may be more appropriate for unusually hard or large cataracts that are possible harder to remove through a smaller incision.

Intrascapular cataract extraction (ICEE) is similar to Extracapsular cataract extraction (ECCE) but with removal of the lens and surrounding lens capsule in one piece.

An evolution of Extracapsular cataract extraction (ECCE), Small incision cataract surgery (SICS) involves expressing the entire lens out of the eye through a self-sealing scleral tunnel wound. An appropriately constructed scleral tunnel is watertight and does not require suturing. Compared to ECCE, SICS leads to shorter operating time and significantly lower costs. In resource poor settings, SICS also has several distinct advantages over phacoemulsification, including shorter operative time, less need for technology, and lower cost.

In laser assisted cataract surgery, a laser (e.g., a femtosecond laser) is used to perform some of the surgical operations that would otherwise be perfumed using surgical blades or other hand-held tools. These surgical operations can include making the initial incisions in the eye, creating an opening in the anterior capsule of the lens to gain access to the cataract, and fragmenting the cloudy lens prior to its removal from the eye.

In this description and the following claims, “regression analysis” is defined as a statistical process for estimating relationships among variables. Regression analysis includes a variety of techniques for modeling and analyzing several variables, including the relationship between a dependent variable and one or more independent variables (or ‘predictors’). Regression analysis can be used to understand how a value of the dependent variable (or ‘criterion variable’) changes when any one of the independent variables is varied, while the other independent variables are held fixed. Regression analysis can estimate the conditional expectation of the dependent variable given the independent variables (i.e., the average value of the dependent variable when the independent variables are fixed).

There are number of different techniques for carrying out regression analysis, including linear regression, ordinary least squares (parametric), Gradient Boosted Decision Trees (GBDT), Random Forest Regression, FastTree regression, FastRank (boosted decision trees) regression, Poisson regression, gradient tree bosting regression, online gradient descent based regression, and neural network based regression.

In this description and the following claims, “linear regression” is defined as an approach for modeling the relationship between a scalar dependent variable y and one or more explanatory variables (or independent variables) denoted X. Linear regression for one explanatory variable can be referred to as simple linear regression. Linear regression for multiple explanatory variables can be referred to as multiple linear regression.

In linear regression, relationships can be modeled using linear predictor models whose unknown model parameters are estimated from the data. For example, the conditional mean of y given the value of X is assumed to be an affine function of X. Less commonly, the median or some other quantile of the conditional distribution of y given X is expressed as a linear function of X. Similar to other regression analysis, linear regression can focuses on the conditional probability distribution of y given X, rather than on the joint probability distribution of y and X (which is instead the domain of multivariate analysis.)

Linear regression has a variety of uses. Linear regression can be used for prediction, forecasting, or error reductions. For example, linear regression can be used to fit a predictive model to an observed data set of y and X values (e.g., cataract surgery effectiveness and cataract surgery parameters). After developing such a model, if an additional value of X is then given without its accompanying value of y, the fitted model can be used to make a prediction of the value of y.

Linear regression models can be fitted using a least squares approach or in other but ways, such as by minimizing the “lack of fit” in some other norm (as with least absolute deviations regression), or by minimizing a penalized version of the least squares loss function as in ridge regression (L²-norm penalty) and lasso (L¹-norm penalty).

Stochastic gradient descent is a gradient descent optimization mechanism for minimizing an objective function that is written as a sum of differentiable functions.

Any of a variety regression analysis techniques including: Gradient Boosted Decision Trees (GBDT), Random Forest Regression, FastTree regression, FastRank (boosted decision trees) regression, Poisson regression, gradient tree bosting regression, online gradient descent based regression, and neural network based regression can be used to model relationships for forecasting cataract surgery effectiveness.

In one aspect, GBDT is implemented using a Multiple Additive Regression Trees (MART) gradient boosting algorithm to forecast cataract surgery effectiveness. MART learns an ensemble of regression trees, which is a decision tree with scalar values in its leaves. The ensemble of trees is produced by computing, in each step, a regression tree that approximates the gradient of the loss function, and adding it to the previous tree with coefficients that minimize the loss of the new tree. The output of the ensemble produced by MART on a given instance is the sum of the tree outputs.

A neural network model can be defined by the structure of its graph (namely, the number of hidden layers and the number of neurons in each hidden layer), the choice of activation function, and the weights on the graph edges. A neural network algorithm tries to learn the optimal weights on the edges based on the training data. In one aspect, a neural network with one hidden layer and a single output neuron is used to forecast cataract surgery effectiveness.

In one aspect, regression techniques are used to model cataract surgery effectiveness from cataract surgery parameters associated with prior cataract surgeries. Cataract surgery effectiveness can include forecasting both refractive outcomes and visual acuity outcomes, recommending a cataract surgery type, and recommending an IOL type and power. Cataract surgery parameters can include any of: patient demographic data, patient pre-operative examination features, patient keratometry, intraocular lens (IOL) features, doctor identifier, surgically induced astigmatism (SIA) value for each doctors, cataract surgery type (e.g., ECCE, SICS, or PHACO), time difference between surgery date and data for which effectiveness is to be forecasted, presence of other eye conditions in patient's eyes, family history, number of prior cataract surgeries, type of cataract, location of IOL placement, patient post-operative sphere, patient post-operative cylinder, patient post-operative Best Corrected Visual Acuity (BCVA), and patient post-operative Uncorrected Visual Acuity (UCVA).

Demographic data can include age, gender, sex, nationality, etc.

Patient pre-operative examination features can include any of: an indication of which eye or eyes are being operated on, uncorrected visual acuity (UCVA), best corrected visual acuity (BCVA), sphere, cylinder, axis, spherical equivalent, uncorrected near vision, corrected near vision, add sphere, and add BCVA.

In this description and in the following claims, “OD” represents an abbreviation for oculus dexter, which is latin for right eye.

In this description and in the following claims, “OS” represents an abbreviation for oculus sinister, which is latin for left eye.

In this description and in the following claims, “sphere” is defined as the correction for nearsightedness or farsightedness being “spherical,” or equal in all meridians of the eye. Sphere can indicate the amount of lens power (e.g., measured in diopters) prescribed to correct nearsightedness or farsightedness. A minus sign (‘−’) can be used to indicate correction of nearsightedness. A plus sign (‘+’) or no sign can be used to indicate correction of farsightedness. Sphere can be measured using a retinoscopy as well as using an auto-refractor.

In this description and in the following claims, “cylinder” is defined as the correction for astigmatism being not spherical. Instead the correction is shaped so one meridian has no added curvature, and the meridian perpendicular to this “no added power” meridian contains the maximum power and lens curvature to correct astigmatism. Cylinder can indicate the amount of lens power to correct astigmatism. A minus sign (‘−’) can be used to indicate correction of nearsighted astigmatism. A plus sign (‘+’) can be used to indicate correction of farsighted astigmatism. Cylinder can be measured using a retinoscopy as well as using an auto-refractor.

In this description and in the following claims, “axis” is defined as orientation of the axis of the cylindrical lens that contains no cylinder power to correct astigmatism. The direction of the axis is measured in degrees anticlockwise from a horizontal line drawn through the center of a pupil (the axis number can be different for each eye) when viewed from the front side of the glasses (i.e., when viewed from the point of view of the person making the measurement). It varies from 1 to 180 degrees. The number 90 can correspond to the vertical meridian of the eye and the number 180 can correspond to the horizontal meridian. Axis can be measured using a retinoscopy as well as using an auto-refractor.

In this description and the following claims, “spherical equivalent” is defined as the spherical power whose focal point coincides with the circle of least confusion of a sphero-cylindrical lens. Hence, the spherical equivalent is equal to the algebraic sum of the value of the sphere and half the cylindrical value.

In this description and in the following claims, “visual acuity” (or “VA”) is defined as clarity of vision. Visual acuity is a measure of the spatial resolution of the visual processing system.

In this description and the following claims, “uncorrected visual acuity (or “UCVA”) is defined as visual acuity without corrective lenses.

In this description and the following claims, “best corrected visual acuity (or “BCVA”) is defined best achievable visual acuity with corrective lenses (e.g., glasses or contacts).

In this description and the following claims, “uncorrected near vision” is defined as visual acuity measured using a small chart held near the patient.

In this description and the following claims, “corrected near vision” is defined as visual acuity measured using a small chart held near the patient with corrective lenses (e.g., glasses or contacts).

In this description and the following claims, “add sphere” is defined as lenses that are bifocal or multifocal. Prescriptions for these types of lenses contain an additional number for sphere.

In this description and the following claims, “add BCVA” is defined as an additional visual acuity value.

Patient keratometry can include any of: K, K1, K2, and axial length. In general, keratometry is a measurement of corneal curvature. Corneal curvature determines the power of the cornea. A keratometer (also known as an ophthalmometer) can be used to measure the curvature of the anterior surface of the cornea

In this description and the following claims, “K1” is defined as a horizontal reading for corneal keratometry. K1 can be in a range from 40 to 50 diopters. In this description and the following claims, “K2” is defined as a vertical reading for corneal keratometry. K2 can be in a range from 40 to 50 diopters. The difference between the horizontal (higher) and vertical (lower) diopter readings approximates the amount of corneal astigmatism, or cylinder correction.

In this description and the following claims, “K” is defined as the average corneal power computed as average of K1 and K2.

In this description and the following claims, “Axial length (AL)” is define as the distance from the anterior corneal surface to the retinal pigment epithelium. AL can be computed using optical or ultrasound techniques.

Intraocular lens (IOL) features can include any of: IOL target refraction, IOL power, lens type, lens A-constant, anterior chamber depth (ACD) constant, corneal radius of curvature, corneal width, corneal height, SRK2 value, and SRKT value.

In this description and the following claims, “IOL target refraction” is defined as target value of sphere that the surgery is intended towards. Emmetropia (0 D) can be the target refraction. If the target of the cataract surgery is to achieve best uncorrected distance and near vision, the target refraction can be in the range of −0.5 D to −1.5 D. In some circumstances, patients may benefit from either a hypermetropic or myopic status postoperatively. Hence, target refraction may be et to a non-zero value in cases where the patient prefers a certain refractive state for either occupational or social needs, including low vision aids. Sometimes the other eye refractive state is considered, as in cases of monovision or anisometropia.

In this description and the following claims, “IOL power” is defined as the power of the lens to be implanted. IOL power indicates the power of the lens that is planned to be used for the surgery.

In this description and the following claims, “Lens Type” is defined as the type of lens intended to be used. Lens type can be selected from among: Aspheric IOL, Foldable, Multifocal, Multifocal Toric, Nonfoldable, Toric. A spherical IOL has a front surface uniformly curved from the center of the lens to its periphery. Aspheric IOLs, on the other hand, match more closely the shape and optical quality of the eye's natural lens, and thereby can provide sharper vision, for example, in low light conditions and for people with larger pupils. Toric IOLs have different powers in different meridians of the lens, and hence can correct astigmatism as well as nearsightedness or farsightedness. Multifocal IOLs contain added magnification in different parts of the lens to expand your range of vision so you can see objects clearly at all distances without glasses or contact lenses. Hence, multifocal IOLs can decrease your need for reading glasses or computer glasses after cataract surgery. Foldable lens are all lenses made of foldable inert material like silicone and acrylic. Foldable lenses can be folded and inserted into the eye through a smaller incision.

In this description and the following claims, “Lens A Constant” is defined as a constant depending on multiple factors, including one or more of: IOL factors (IOL type, IOL material, and IOL position), surgeon factors (incision technique and placement of incision), corneal power (K), axial length measurement adjustments, and adjustment for the manner of carrying out biometry.

In this description and the following claims, “ACD (anterior chamber depth) constant” can be estimated from 0.62467×A−68.747, where A is the Lens A Constant.

In this description and the following claims, “Corneal Radius of Curvature (R)” is defined as the radius of curvature of the cornea. R can be computed as R=337.5˜K.

In this description and the following claims, “Corneal Width (W)” is defined as width of the cornea. W can be computer as W=−5.41+(0.58412×LCOR)+(0.098×K), where LCOR is the corrected axial length with an adjustment for smaller eyes.

SRK2 value and SRKT values are derived from formulas used to estimate power of the IOL.

A doctor identifier can be used to link features related to a doctor, for example, SIA, to surgeries performed by the doctor.

The presence of other eye conditions can include the presence of any of: post refractive surgery, uveitis, Glaucoma eyes, eyes with corneal problems like scars or keratoconus, post Vitreo-retinal surgery, Vitrectomized eyes, silicon oil filled eyes, Dislocated lens, or eyes with pseudoexfoliation.

Cataract type can include any of: total cataract, immature cataract, nuclear cataract, Cortical spoking cataract, Posterior subcapsular cataract, combination, or grade of cataract (e.g., 1 to 5).

IOL placement location can include any of: into the bag, over the iris, or under the bag.

In general, a predictive model for cataract surgery effectiveness can be formulated from and trained on cataract surgery parameters from previously conducted cataract surgeries. Cataract surgery parameters can be obtained for hundreds or even thousands of cataract surgeries.

Domain knowledge can be used to preprocess data by transforming some categorical features into binary features. A categorical feature can be a column in the data which takes any of a number of unique values. For example, topography machine used for the surgery is a categorical feature may take three values (Orbscan, Galilei, or Oculyzer). A binary feature is one that takes two values (0 or 1). Some classification and regression models are not well suited to handing categorical variables. As such, categorical variables can be converted to binary indicator variables.

Surgery type can be a categorical variable with three unique values: ECCE, SICS, and PHACO. The categorical feature can be converted into three corresponding binary variables, one each for each of the unique values. Thus, there are now three features to represent surgery name. For an instance of surgery parameters, one of the three features has a value of 1 while others have a value of 0.

For some categorical features, domain knowledge can indicate that there may not be one feature per unique value. For example, some unique values have limited value when forecasting cataract surgery effectiveness. For example, a categorical feature called Retina examination may have any number of different values indicating abnormality, such as, for example, “Chorioretinal Atrophy”, “Familial Exudative Vitreo-retinopathy (FEVR)”, “Barrage laser done”, “Retinal pigment epithelium (RPE) atrophy”, “Tilted disc with temporal pallor” or may have the value “normal”. When forecasting cataract surgery effectiveness for patient, the specific abnormality type may be of limited value. As such, a categorical feature for Retina examination can be transformed into a single binary feature for Retina examination. The binary feature can take values of 0 or 1 for abnormal and normal or vice versa

To further fill-in pre-operative examination results, average values can be used to impute missing values for numerical features. Most frequent values can be used to impute missing value for categorical features.

Multiple regression approaches can be evaluated.

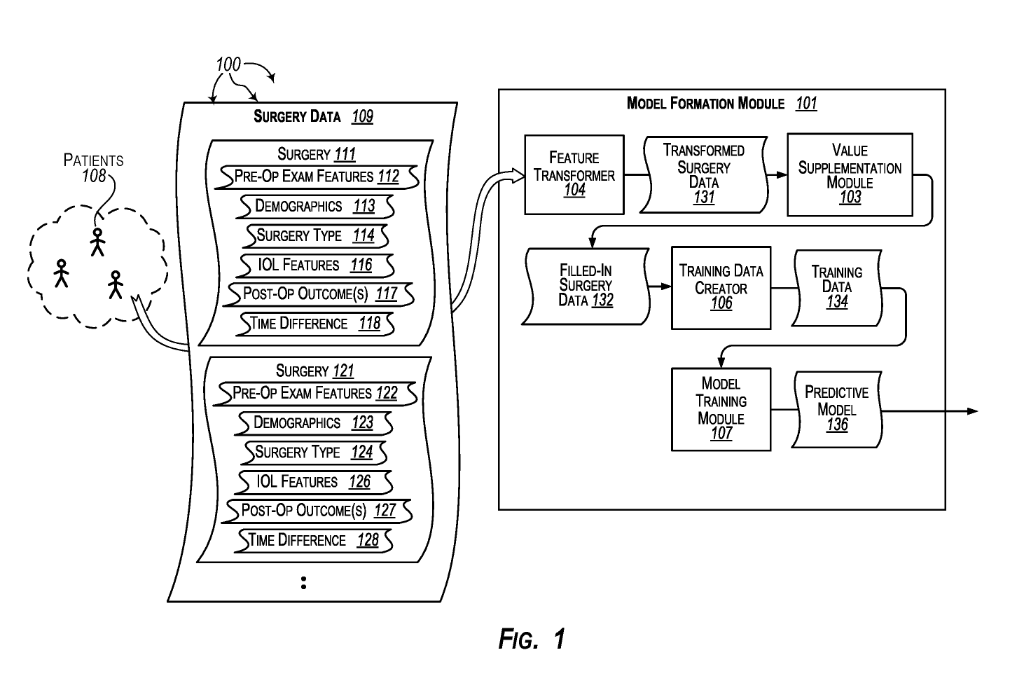

FIG. 1 illustrates an example computer architecture 100 that facilitates formulating and training a predictive model to provide personalized refractive surgery recommendations for eye patients. Referring to FIG. 1, computer architecture 100 includes model formulation module 101 and patients 108. Model formulation module 101 and information technology resources storing examination data for patients 108 can be connected to (or be part of) a network, such as, for example, a system bus, a Local Area Network (“LAN”), a Wide Area Network (“WAN”), and even the Internet. Accordingly, model formulation module 101 and information technology resources storing examination data for patients 108 as well as any other connected computer systems and their components can create and exchange message related data (e.g., Internet Protocol (“IP”) datagrams and other higher layer protocols that utilize IP datagrams, such as, Transmission Control Protocol (“TCP”), Hypertext Transfer Protocol (“HTTP”), Simple Mail Transfer Protocol (“SMTP”), Simple Object Access Protocol (SOAP), etc. or using other non-datagram protocols) over the network.

Model formulation module 101 further includes feature transformer 102, value supplementation module 103, training data creator 106, and model training module 107. Feature transformer 102 is configured to transform values for categorical features into corresponding values for binary features. Value supplementation module 103 is configured to fill in missing values transformed surgery data. Training data creator 106 is configured to generated training data from filled-in surgery data. Model training module 107 is configured to train and formulate predictive models from training data.

Each of patients 108 can be a patient that has undergone cataract surgery. The cataract surgeries can be performed by an eye practitioner or by a plurality of different eye practitioners. Surgery data for each cataract surgery can be stored on information technology resources, for example, at an eye practitioner's office, at a hospital, in a cloud, etc.

Appropriate health care and/or privacy regulations (depending on country) can be followed to collect the surgery data for each cataract surgery. The collected surgery data can be combined into surgery data 109. Surgery data 109 can include at least a subset of surgery parameters for each of plurality (e.g., hundreds or thousands) of previously performed cataract surgeries. However, any personally identifiable information for eye patients can be omitted from surgery data 109. Surgery data 109 may be stored at a storage device (e.g., locally, on cloud resources, etc.) under control of an entity that creates predictive models. Surgery data 109 can be stored in accordance with appropriate health care and/or privacy regulations.

More specifically, surgery data 109 incudes, for each cataract surgery, at least a subset of surgery parameters from among any of: patient demographic data, patient pre-operative examination features, patient keratometry, intraocular lens (IOL) features, doctor identifier identifying a doctor, surgically induced astigmatism (SIA) value for the doctor, cataract surgery type (e.g., ECCE, SICS, or PHACO), time difference between surgery date and date for which effectiveness was evaluated, presence of other eye conditions in patient's eyes, family history, number of prior cataract surgeries, type of cataract, location of IOL placement, patient post-operative sphere, patient post-operative cylinder, patient post-operative Best Corrected Visual Acuity (BCVA), and patient post-operative Uncorrected Visual Acuity (UCVA).

In one aspect, surgery parameters for at least some previously performed cataract surgery include: demographic data, at least some of the described patient pre-operative examination features, a cataract surgery type, at least some of the described IOL features, at least one post-operative outcome (e.g., related to visual refraction or visual acuity), and a time difference (e.g., in days) between the cataract surgery and a post-operative exam.

For example, examination data 109 includes surgery 111, surgery 121, etc. Surgery 111 includes pre-op exam features 112, demographics 113, surgery type 114, IOL features 116, post-op outcome(s) 117, and time difference 118. Similarly, surgery 121 includes pre-op exam features 122, demographics 123, surgery type 124, IOL features 126, post-op outcome(s) 127, and time difference 128. Surgery parameters for other previously performed cataract surgeries can grouped in less detail, in essentially the same detail, or in more detail. Thus, in general, there can be some variability in how surgery parameters are grouped for any previously performed surgeries. In one aspect, a group of surgery parameters are specifically selected so that particular parameters are used as influencing variables when forecasting cataract surgery effectiveness.

Model formulation module 101 can access surgery data 109. Model formulation module 101 can use surgery data 109 to formulate predictive model 136. Predictive model 136 can forecast cataract surgery effectiveness for an eye patient. Cataract surgery effectiveness can include forecasting both refractive outcomes and visual acuity outcomes, recommending a cataract surgery type, and recommending an IOL type and power. Using predictive model 136 forecasted outcomes for different prospective patients can be determined. Prospective patients can be ranked based on their probability of having a positive outcome from cataract surgery.

Surgery data 109 can be initially received at feature transformer 102. Feature transformer 102 can transform one or more (but not necessarily all) categorical features in surgery data 109 into corresponding sets of binary features. Feature transformer 102 can output transformed surgery data 131.

Value supplementation module 103 receives transformed surgery data 131 as input. Value supplementation module 103 imputes values into transformed surgery data 131 for missing values. Value supplementation module 103 can impute average values for missing values for numeric features. Average values can be determined from other values for the same numeric features in other surgery data. Value supplementation module 103 can impute (e.g., most) frequently used values for missing values for categorical features. Frequently used values can be determined from other values for the same categorical features in other surgery data.

Value supplementation module 103 outputs filled-in surgery data 132. Filled-in surgery data 132 includes transformed surgery data with a number of (and potentially all) missing values in surgery data 109 replaced with average or frequently used values. Training data creator 106 receives filled-in surgery data 132 as input.

Training data creator 106 uses filled-in surgery data 132 to create training data 134. Training data 134 can include different forms and types of training data used to train different types of regression models. Training data creator 106 outputs training data 134 including a plurality of training data entries.

Model training module 107 receives training data 134 as input. Model training module 107 uses training data 134 to formulate and train predictive model 136. Model training module 107 can train on multiple regression models, such as, for example, Gradient Boosted Decision Trees (GBDT), Random Forest Regression, FastTree regression, FastRank (boosted decision trees) regression, Poisson regression, gradient tree bosting regression, etc.

In one aspect, a Multiple Additive Regression Trees (“MART”) gradient boosting algorithm learns an ensemble of regression trees, which is a decision tree with scalar values in its leaves. Functions produced by a regression tree can be piece-wise constant functions. The ensemble of trees is produced by computing, in each part, a regression tree that approximates the gradient of the loss function, and adding it to the previous tree with coefficients that minimize the loss of the new tree. The output of the ensemble produced by MART on a given instance is the sum of the tree outputs. In case of a regression problem, the output is the predicted value of the function.

In general, medical practitioners can use predictive model 136 to automatically forecast cataract surgery effectiveness for patients, including predicting both refractive outcomes (cylinder and sphere) and visual acuity outcomes (UCVA and BCVA), recommending a cataract surgery type, and recommending an IOL type and power. Predictive model 136 can be offered to medical practitioners as a Web API, as an application on the web, as a SaaS offering, as an application on mobiles or medical devices, integrated into medical device hardware, or any number of other platforms.

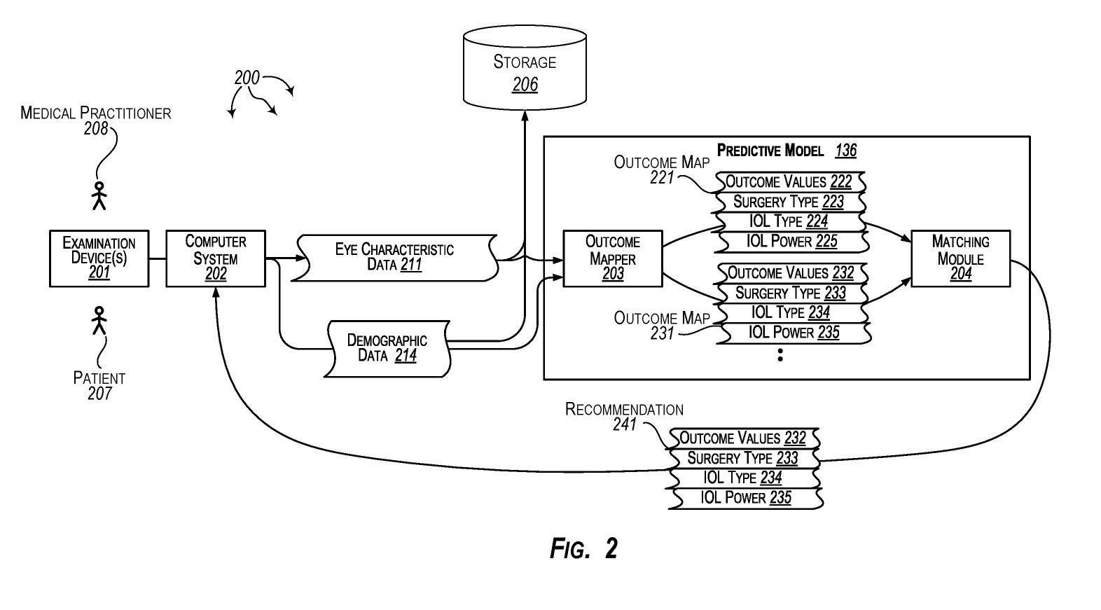

Turning to FIG. 2, FIG. 2 illustrates an example computer architecture that facilitates forecasting cataract surgery effectiveness for eye patients. Referring to FIG. 2, computer architecture 200 includes examination device(s) 201, computer system 202, storage 206, and predictive model 136. Examination device(s) 201, computer system 202, storage 206, and predictive model 136 can be connected to (or be part of) a network, such as, for example, a system bus, a Local Area Network (“LAN”), a Wide Area Network (“WAN”), and even the Internet. Accordingly, examination device(s) 201, computer system 202, storage 206, and predictive model 136 as well as any other connected computer systems and their components can create and exchange message related data (e.g., Internet Protocol (“IP”) datagrams and other higher layer protocols that utilize IP datagrams, such as, Transmission Control Protocol (“TCP”), Hypertext Transfer Protocol (“HTTP”), Simple Mail Transfer Protocol (“SMTP”), Simple Object Access Protocol (SOAP), etc. or using other non-datagram protocols) over the network.

Predictive model 136 can be run in system memory, such as, for example, system memory of computer system 202 or system memory within cloud resources.

Medical practitioner 208 can perform an eye examination on patient 207. As part of the eye examination, medical practitioner 208 can use examination device(s) 201 to measure and record values for eye characteristics of patient 207. The eye examination may or may not be related to a previous indication of the patient being a candidate for cataract surgery. The eye examination can take place in any of a variety of locations, in the patient 207's house, in a doctor's office, in an ambulance, at a hospital, at a clinic, indoors, outdoors, etc. Medical practitioner 208 can be more or less skilled with respect to diagnosing and treating cataracts, ranging from an emergency medical technician (EMT), to a paramedic, to a nurse, to a general practice doctor, to a doctor specializing in diseases of the eye. In one aspect, medical practitioner 208 is checking the general health of patient 207 (e.g., during a physical examination) and is not specifically examining patient 207 with respect to diagnosing or treating cataracts or other eye diseases.

In one aspect, medical practitioner 208 uses examination device(s) to measure one or more of: uncorrected visual acuity (UCVA), best corrected visual acuity (BCVA), sphere, cylinder, axis, spherical equivalent, uncorrected near vision, corrected near vision, add sphere, add BCVA, K1, K2, K, and Axial Length for each of patient 107's eyes. During measurement of patient 107's eye characteristics, medical practitioner 108 can determine that patient 107's vision in at least one eye is at least partially impaired due to cataract. Values for various measured eye characteristics of patient 207 can be recorded in eye characteristics data 211.

In one aspect, device(s) 201 include a device for generating a topographical map of patient 207's eyes. Eye characteristic data 211 can be derived at least in part from the topographical map.

Demographic data for patient 207 (e.g., age, gender, etc.) can be recorded in demographic data 214 at computer system 202. Eye characteristic data 211 and demographic data 214 can also be stored in storage 206 (which may be local at eye practitioner 208's office, at a remote storage location, in the cloud, etc.).

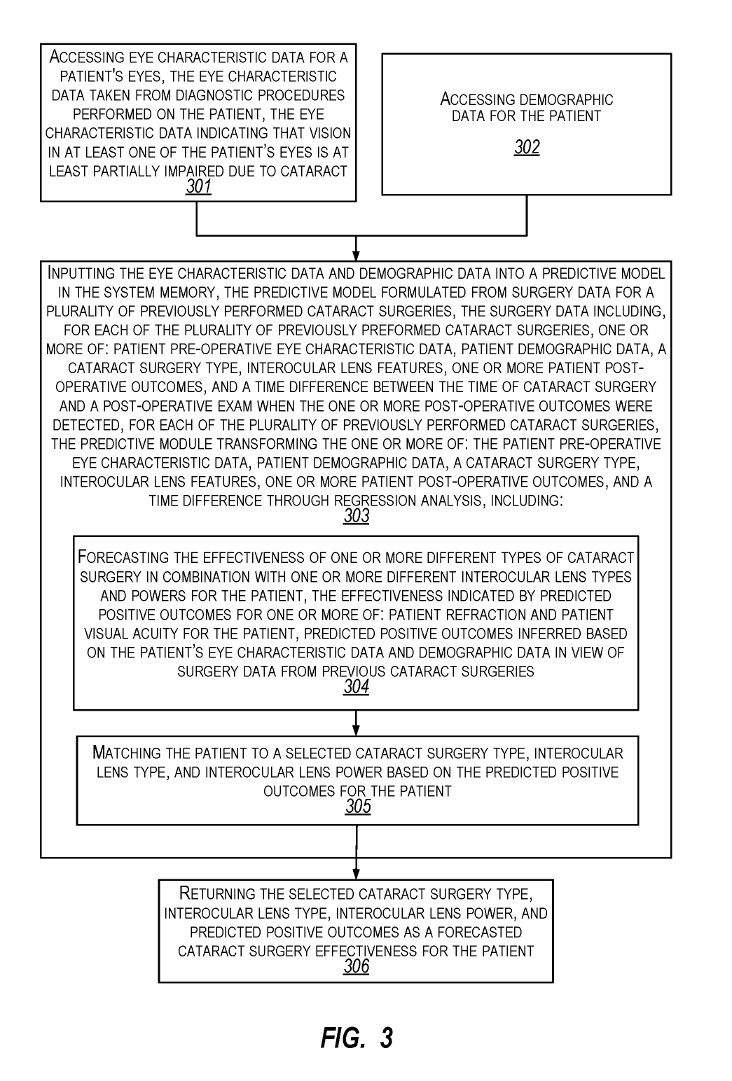

FIG. 3 illustrates a flow chart of an example method 300 for forecasting cataract surgery effectiveness for eye patients. Method 300 will be described with respect to the components and data of computer architecture 200.

Method 300 includes accessing eye characteristic data for the patient's eyes, the eye characteristic data taken from diagnostic procedures performed on the patient, the eye characteristic data indicating that vision in at least one of the patient's eyes is at least partially impaired due to cataract (301). For example, predictive model 136 can access eye characteristic data 211. Characteristics data can be send to predictive model due to medical practitioner 208 determining that vision in at least one of the patient 207's eyes is at least partially impaired due to cataract. Method 300 includes accessing demographic data for the patient (302). For example, predictive model 136 can access demographic data 214.

Method 300 includes input the eye characteristic data and demographic data into a predictive model in the system memory, the predictive model formulated from surgery data for a plurality of previously performed cataract surgeries, the surgery data including, for each of the plurality of previous performed cataract surgeries, one or more of: patient pre-operative eye characteristic data, patient demographic data, a cataract surgery type, interocular lens features, one or more patient post-operative outcomes, and a time difference between the time of cataract surgery and a post-operative exam when the one or more post-operative outcomes were detected, for each of the plurality of previously performed cataract surgeries, the predictive model transforming the one or more of: the patient pre-operative eye characteristic data, the patient demographic data, the cataract surgery type, the interocular lens features, the one or more patient post-operative outcomes, and the time difference through regression analysis (303). For example, eye characteristic data 211 and demographic data 214 can be input to outcome mapper 203.

Transforming the one or more of: the patient pre-operative eye characteristic data, the patient demographic data, the cataract surgery type, the interocular lens features, the one or more patient post-operative outcomes, and the time difference through regression analysis includes forecasting the effectiveness of one or more different types of cataract surgery in combination with one or more different interocular lens types and powers for the patient, the effectiveness indicated by predicted positive outcomes for one or more of: patient refraction and patient visual acuity for the patient, predicted positive outcomes inferred based on the patient's eye characteristic data and demographic data in view of surgery data from previous cataract surgeries (304). For example, acuity mapper 203 can predict outcome map 221, outcome map 231, etc. for patient 207. The ellipsis below outcome map 231 represents that outcome mapper 203 can predicting one or more other outcome maps for other combinations of cataract surgery type, interocular lens type, and interocular lens power.

Each of outcome map 221, outcome map 231, etc. is inferred for a corresponding type of refractive surgery based on eye characteristic data 211 and demographic data 214. For example, outcome map 221 is inferred for surgery type 223, interlocular lens type 224, and interocular lens power 225. Outcome map 231 is inferred for surgery type 233, interlocular lens type 234, and interocular lens power 235. Outcome map 221 predicts post-operative outcome values 222 for patient 207 at one or more post-operative time periods (e.g., one day, one week, and one month), if patient 207 were to surgery type 223 with interlocular lens type 224 and interocular lens power 225. Similarly, outcome map 231 predicts post-operative outcome vales 232 for patient 207 at one or more post-operative time periods (e.g., one day, one week, and one month) if patient 207 were to surgery type 233 with interlocular lens type 234 and interocular lens power 235.

Transforming the one or more of: the patient pre-operative eye characteristic data, the patient demographic data, the cataract surgery type, the interocular lens features, the one or more patient post-operative outcomes, and the time difference through regression analysis includes matching the patient to a selected cataract surgery type, interocular lens type, and interocular lens power based on the predicted positive outcomes for the patient (305). For example, matching module 204 can match patient 207 to outcome map 231 based on outcome values 232. Outcome values 232 can indicate an improved outcome (e.g., increased refractive improvement and/or visual acuity improvement) over time relative to outcome values 222 and outcome values in any other outcome maps.

Method 300 includes returning the selected cataract surgery type, interocular lens type, interocular lens power, and predicted positive outcomes as a forecasted cataract surgery effectiveness for the patient (306). For example, matching module 204 can return recommendation 241, including predicted outcome values 232, surgery type 233, interlocular lens type 234, and interocular lens power 235, to computer system 202 as a forecasted cataract surgery effectiveness for patient 207. Computer system 202 can present recommendation 241 at a user-interface screen. Medical practitioner 208 and patient 207 can view recommendation 241 at the user-interface screen. A hard copy of recommendation 241 can also be printed for medical practitioner 208 and/or patient 207.

In one aspect, predictive model 136 is used to predict if a patient with have a specified predicted outcome. For example, predictive model 136 can be used to predict if patient 207's post-operative vision is to be within +/−0.5 sphere or not. Predictive model 136 can also be used to predict if patient 207's post-operative vison is to have SIA greater than 1.

Examination device(s) 201, computer system 202, and predictive model 136 can operate in essentially real-time to forecast cataract surgery effectiveness. Data from examination device(s) 201 are automatically fed into computer system 202. In turn, computer system 202 automatically sends eye characteristic data 211 and demographic data 214 to predictive model 136. Predictive model 136 automatically generates and returns recommendation 241 back to computer system 202. Computer system 202 then automatically displays/prints recommendation 213.

Medical practitioner 208 can follow-up with additional examinations of patient 107 or can recommend a more skilled medical practitioner. For example, a general practice doctor may recommend another document specializing in diseases of the eye.

Predictive model 136 can also be integrated into other medical diagnostic equipment, such as, for example, a retinoscope, a retinal camera, an autorefractor, a keratometer, an ophthalmometer, a phoropter, a tonometer, a lensometer, an indirect ophthalmoscope, a direct ophthalmoscope, slit lamp, etc. included in examination device(s) 201. In response to measuring eye characteristics for a patient, medical diagnostic equipment can automatically use predictive model 136 to also forecast cataract surgery effectiveness for the patient. The diagnostic equipment can include computing resources for utilizing predictive model 136 locally or can interact with predictive model 136 via network communication.

In one aspect, predictive model 136 is installed on local computing resources and/or hardware resources (e.g., programed into an ASIC or FPGA) within a medical device. The medical device utilizes predictive model 136 entirely locally to forecast cataract surgery effectiveness for patients internally. For example, computer system 202, predictive model 136, and storage 206, can be contained in one of examination device(s) 201. In response to sensing/measuring eye characteristics for patient 207, the examination device 201 can internally transfer the sensed/measured eye characteristics to predictive model 136 to locally forecast cataract surgery effectiveness for patient 207.

In another aspect, a medical device accesses predictive model 136 via a cloud based service. The medical device calls the cloud based service with relevant parameters, the cloud based service uses predictive model 136 to forecast cataract surgery effectiveness, and the cloud based services returns a forecasted cataract surgery effectiveness. A returned forecasted effectiveness of cataract surgery for a patient can be returned to the medical device or another electronic device, for example, a mobile phone, in essentially real-time.

In one aspect, predictive model 136 is used by different devices and/or computer systems at different locations to forecast cataract surgery effectiveness for a plurality of patients, including patient 207. The plurality of patients can be spread across a geographic area with limited medical resources to perform cataract surgeries. Predicted outcomes for patients collected from different physical locations can be stored in a centralized database. Predicted outcomes can be used to rank patients relative to one another. For example, outcome values 232 can be used to rank patient 207 relative to other patients based on their outcome values.

In one aspect, patients are grouped using different factors into a plurality of different groups. Factors can include one or more of: ranks, condition severity, surgery prognosis, etc. as well as combinations thereof. For example, one group can include patients with very high grade of cataract and very high chances of getting almost perfect eyes if operated within a shorter time frame. Another group can include patients with very high grade of cataract but with low chances of much improvement. Additional groups can include patients with different combinations of condition severity and/or surgery prognosis. Such grouping of patients can be performed using clustering.

Limited medical resources can be prioritized for use by higher ranked patients and/or patients in specified groups. For example, medical resources can be allocated for patients with a higher likelihood of sight improvement or having a likelihood of more significant sight improvement via cataract surgery over other patients with a lower likelihood of sight improvement or having a likelihood of less significant sight improvement via cataract surgery. Patients can be re-evaluated from time to time to determine if outcome values have changed. Accordingly, ranking and/or grouping patients allows governmental and aid entities to better allocate medical resources to those in need and/or to those that would get increased benefits.

If patient 207 has cataract surgery, an eye surgeon can conduct one or more post-operative exams, including measuring post-operative refraction values and measuring post-operative visual acuity values. Post-operative exams can occur, one day after cataract surgery, one week after cataract surgery, and one month after cataract surgery. Eye characteristic data 211, demographic data 214, surgery type 233, IOL type 234, IOL power 235, measured refraction values, and measured visual acuity values can be provided as feedback to model formulation module 106 and/or are included in surgery data 109. Eye characteristic data 211, demographic data 214, surgery type 233, IOL type 234, IOL power 235, measured refraction values, and measured visual acuity values can be used to facilitate further training and refinement of predictive model 136.

Accordingly, aspects of the invention can be used forecast cataract surgery effectiveness for a patient given eye characteristic data and demographic details. Regression models can be used to effectively forecast post-operative refraction values and visual acuity values from demographics and eye examination features. Other machine learning methods and other forms of classification and regression can also be used.

In some aspects, a computer system comprises one or more hardware processors and system memory. The one or more hardware processors are configured to execute instructions stored in the system memory to forecast cataract surgery effectiveness for a patient.

The one or more hardware processors execute instructions stored in the system memory to access eye characteristic data for the patient's eyes. The eye characteristic data is taken from diagnostic procedures performed on the patient. The eye characteristic data indicates that vision in at least one of the patient's eyes is at least partially impaired due to cataract. The one or more hardware processors execute instructions stored in the system memory to access demographic data for the patient.

The one or more hardware processors execute instructions stored in the system memory to input the eye characteristic data and demographic data into a predictive model in the system memory. The predictive model is formulated from surgery data for a plurality of previously performed cataract surgeries. The surgery data includes, for each of the plurality of previous performed cataract surgeries, one or more of: patient pre-operative eye characteristic data, patient demographic data, a cataract surgery type, interocular lens features, one or more patient post-operative outcomes, and a time difference between the time of cataract surgery and a post-operative exam when the one or more post-operative outcomes were detected. For each of the plurality of previously performed cataract surgeries, the predictive model transforms the one or more of: the patient pre-operative eye characteristic data, the patient demographic data, the cataract surgery type, the interocular lens features, the one or more patient post-operative outcomes, and the time difference through regression analysis.

The one or more hardware processors execute instructions stored in the system memory to forecast the effectiveness of one or more different types of cataract surgery in combination with one or more different interocular lens types and powers for the patient. The effectiveness for each of one or more different types of cataract surgeries in combination with one or more different interocular lens types and powers is indicated by predicted positive outcomes for one or more of: patient refraction and patient visual acuity for the patient. Predicted positive outcomes are inferred based on the patient's eye characteristic data and demographic data in view of surgery data from previous cataract surgeries.

The one or more hardware processors execute instructions stored in the system memory to match the patient to a selected cataract surgery type, interocular lens type, and interocular lens power based on the predicted positive outcomes for the patient. The one or more hardware processors execute instructions stored in the system memory to return the selected cataract surgery type, interocular lens type, interocular lens power, and predicted positive outcomes as a forecasted cataract surgery effectiveness for the patient.

Computer implemented methods for performing the executed instructions to (e.g., automatically) forecast cataract surgery effectiveness for an eye patient are also contemplated. Computer program products storing the instructions, that when executed by a processor, cause a computer system to (e.g., automatically) forecast cataract surgery effectiveness for an eye patient are also contemplated.

The present described aspects may be implemented in other specific forms without departing from its spirit or essential characteristics. The described aspects are to be considered in all respects only as illustrative and not restrictive. The scope is, therefore, indicated by the appended claims rather than by the foregoing description. All changes which come within the meaning and range of equivalency of the claims are to be embraced within their scope.

What is claimed:

1. A computer system, the computer system comprising:
one or more hardware processors;
system memory coupled to the one or more hardware processors, the system memory storing instructions that are executable by the one or more hardware processors;
the one or more hardware processors executing the instructions stored in the system memory to forecast cataract surgery effectiveness for a patient, including the following:
access eye characteristic data for the patient's eyes, the eye characteristic data taken from diagnostic procedures performed on the patient, the eye characteristic data indicating that vision in at least one of the patient's eyes is at least partially impaired due to cataract;
access demographic data for the patient;
input the eye characteristic data and demographic data into a predictive model in the system memory, the predictive model formulated from surgery data for a plurality of previously performed cataract surgeries, the surgery data including, for each of the plurality of previous performed cataract surgeries, one or more of: patient pre-operative eye characteristic data, patient demographic data, a cataract surgery type, interocular lens features, one or more patient post-operative outcomes, and a time difference between the time of cataract surgery and a post-operative exam when the one or more post-operative outcomes were detected, for each of the plurality of previously performed cataract surgeries, the predictive model transforming the one or more of: the patient pre-operative eye characteristic data, the patient demographic data, the cataract surgery type, the interocular lens features, the one or more patient post-operative outcomes, and the time difference through regression analysis to:
forecast the effectiveness of one or more different types of cataract surgery in combination with one or more different interocular lens types and powers for the patient, the effectiveness indicated by predicted positive outcomes for one or more of: patient refraction and patient visual acuity for the patient, predicted positive outcomes inferred based on the patient's eye characteristic data and demographic data in view of surgery data from previous cataract surgeries;
match the patient to a selected cataract surgery type, interocular lens type, and interocular lens power based on the predicted positive outcomes for the patient; and
return the selected cataract surgery type, interocular lens type, interocular lens power, and predicted positive outcomes as a forecasted cataract surgery effectiveness for the patient.

2. The computer system of claim 1, wherein the one or more hardware processors executing the instructions stored in the system memory to access eye characteristic data for the patient's eyes comprises the one or more hardware processors executing the instructions stored in the system memory to access one or more of: uncorrected visual acuity (UCVA), uncorrected near vision, corrected near vision, best-corrected visual acuity (BCVA) with corrective lenses, sphere, cylinder and axis for the patient.

3. The computer system of claim 1, further comprising the one or more hardware processors executing the instructions stored in the system memory to transform surgery parameters for a subset of the plurality of other patients through regression analysis to predict surgery parameters for the patient, the surgery parameters predicted from surgery parameters used in other cataract surgeries.

4. The computer system of claim 1, wherein the one or more hardware processors executing the instructions stored in the system memory to predict positive outcomes for one or more of: patient refraction and patient visual acuity for the patient comprises the one or more hardware processors executing the instructions stored in the system memory to predict post-operative sphere, post-operative cylinder, post-operative uncorrected visual acuity (UCVA), and post-operative best-corrected visual acuity (BCVA) for the patient.

5. The computer system of claim 1, further comprising the one or more hardware processors executing the instructions stored in the system memory to perform one or more of:
replace a missing value for a feature in the other patient data with an average value for the feature, the average value for the feature averaged from other values for the feature contained in the patient data; and
replace a missing value for feature in the other patient data with a most frequently used value for the feature contained in the patient data.

6. The computer system of claim 1, wherein the predictive model being formulated from other patient data comprises the predictive model being formulated based on one or more categorical features contained in the patient data, each categorical feature having an enumerated plurality of specified possible values.

7. The computer system of claim 1, further comprising the one or more hardware processors executing the instructions stored in the system memory to transform a categorical feature, from among the one or more categorical features, into a corresponding plurality of binary features collectively representing the categorical feature, each corresponding binary feature representing one of the enumerated plurality of specified possible values for the categorical feature; and
wherein the predictive model being formulated based on one or more categorical features comprises the predictive model being formulated based on the corresponding plurality of binary features.

8. A method for use at a computer system, the method for forecasting cataract surgery effectiveness for a patient, the method comprising the following:
accessing eye characteristic data for the patient's eyes, the eye characteristic data taken from diagnostic procedures performed on the patient, the eye characteristic data indicating that vision in at least one of the patient's eyes is at least partially impaired due to cataract;
accessing demographic data for the patient;
inputting the eye characteristic data and demographic data into a predictive model in the system memory, the predictive model formulated from surgery data for a plurality of previously performed cataract surgeries, the surgery data including, for each of the plurality of previous performed cataract surgeries, one or more of: patient pre-operative eye characteristic data, patient demographic data, a cataract surgery type, interocular lens features, one or more patient post-operative outcomes, and a time difference between the time of cataract surgery and a post-operative exam when the one or more post-operative outcomes were detected, for each of the plurality of previously performed cataract surgeries, the predictive model transforming the one or more of: the patient pre-operative eye characteristic data, the patient demographic data, the cataract surgery type, the interocular lens features, the one or more patient post-operative outcomes, and the time difference through regression analysis to:
forecasting the effectiveness of one or more different types of cataract surgery in combination with one or more different interocular lens types and powers for the patient, the effectiveness indicated by predicted positive outcomes for one or more of: patient refraction and patient visual acuity for the patient, predicted positive outcomes inferred based on the patient's eye characteristic data and demographic data in view of surgery data from previous cataract surgeries;
matching the patient to a selected cataract surgery type, interocular lens type, and interocular lens power based on the predicted positive outcomes for the patient; and
returning the selected cataract surgery type, interocular lens type, interocular lens power, and predicted positive outcomes as a forecasted cataract surgery effectiveness for the patient.

9. The method of claim 8, wherein the predictive model being formulated from other patient data comprises the predictive model being formulated based on one or more categorical features contained in the patient data, each categorical feature having an enumerated plurality of specified possible values;
further comprising transforming a categorical feature, from among the one or more categorical features, into a corresponding plurality of binary features collectively representing the categorical feature, each corresponding binary feature representing one of the enumerated plurality of specified possible values for the categorical feature; and
wherein the predictive model being formulated based on one or more categorical features comprises the predictive model being formulated based on the corresponding plurality of binary features.

10. A computer program product for use at a computer system, the computer program product for implementing a method for forecasting cataract surgery effectiveness for a patient, the computer program product comprising one or more hardware storage devices having stored thereon computer-executable instructions that, when executed at a processor, cause the computer system to perform the method, including the following:
access eye characteristic data for the patient's eyes, the eye characteristic data taken from diagnostic procedures performed on the patient, the eye characteristic data indicating that vision in at least one of the patient's eyes is at least partially impaired due to cataract;
access demographic data for the patient;
input the eye characteristic data and demographic data into a predictive model in the system memory, the predictive model formulated from surgery data for a plurality of previously performed cataract surgeries, the surgery data including, for each of the plurality of previous performed cataract surgeries, one or more of: patient pre-operative eye characteristic data, patient demographic data, a cataract surgery type, interocular lens features, one or more patient post-operative outcomes, and a time difference between the time of cataract surgery and a post-operative exam when the one or more post-operative outcomes were detected, for each of the plurality of previously performed cataract surgeries, the predictive model transforming the one or more of: the patient pre-operative eye characteristic data, the patient demographic data, the cataract surgery type, the interocular lens features, the one or more patient post-operative outcomes, and the time difference through regression analysis to:
forecast the effectiveness of one or more different types of cataract surgery in combination with one or more different interocular lens types and powers for the patient, the effectiveness indicated by predicted positive outcomes for one or more of: patient refraction and patient visual acuity for the patient, predicted positive outcomes inferred based on the patient's eye characteristic data and demographic data in view of surgery data from previous cataract surgeries;
match the patient to a selected cataract surgery type, interocular lens type, and interocular lens power based on the predicted positive outcomes for the patient; and
return the selected cataract surgery type, interocular lens type, interocular lens power, and predicted positive outcomes as a forecasted cataract surgery effectiveness for the patient.
